(19) United States
(12) Patent Application Publication
(10) Pub. No.: US 2025/0032183 A1
(43) Pub. Date: Jan. 30, 2025

(54) METHODS AND APPARATUS FOR EVALUATING AND TREATING DISEASED VESSELS

(71) Applicant: Gravity Medical Technology, Inc., Carlsbad, CA (US)

(72) Inventors: Shashvat M. Desai, Phoenix, AZ (US); Kunakorn Atchaneeyasakul, Renton, WA (US); Ashutosh P. Jadhav, Paradise Valley, AZ (US)

(21) Appl. No.: 18/916,099

(22) Filed: Oct. 15, 2024

Related U.S. Application Data
(63) Continuation of application No. PCT/US2023/019598, filed on Apr. 24, 2023.
(60) Provisional application No. 63/334,295, filed on Apr. 25, 2022.

(51) Int. Cl. A61B 18/14; A61B 17/00; A61B 17/22; A61B 17/221; A61B 18/00; A61N 1/36

(52) U.S. Cl. CPC A61B 18/1492; A61B 17/221; A61N 1/36; A61B 2017/00026; A61B 2017/00057; A61B 2017/22084; A61B 2017/2215; A61B 2018/00214; A61B 2018/0041; A61B 2018/00642; A61B 2018/00875; A61B 2018/00982

(57) ABSTRACT

A device for treating a thrombus or spasm or otherwise diseased vessel in a patient may include an elongate shaft with a lumen. An electro-pharmaceutical unit may be coupled to the proximal end of the elongate shaft, and that is configured to facilitate evaluation and treatment of the thrombus or spasm or other disease in the vessel. An expandable cage may be coupled to the distal end of the elongate shaft. The cage may have a collapsed configuration for delivery through a vessel to the thrombus, spasm or diseased area of the vessel, and an expanded configuration for enmeshing the thrombus or supporting the spasm or diseased vessel. An electrical element may be coupled to the expandable cage, and the electrical element may be configured to sense a characteristic of the thrombus or the diseased vessel or to provide a treatment to the thrombus, spasm, or diseased vessel.

### CLAIM OF PRIORITY

This patent application is a U.S. National Stage Application under 35 U.S.C. 371 from International Application No. PCT/US2023/019598 filed Apr. 24, 2023, and published as WO 2023/211819 on Nov. 2, 2023, which claims the benefit of priority of U.S. Provisional Patent Application No. 63/334,295 (Attorney Docket No. 5543.003 PRV) filed on Apr. 25, 2022, each of which are incorporated herein by reference in their entirety.

### CROSS-REFERENCE TO RELATED PATENT DOCUMENTS

This patent application is also related to U.S. patent application Ser. No. 17/336,791 now U.S. Pat. No. 11,291,463; the entire contents of which are incorporated herein by reference.

### BACKGROUND

Occlusion of a blood vessel is often caused by a clot (also referred to herein as thrombus) and this may be referred to as a thromboembolic event, and can result in disorders such as stroke, pulmonary embolism, peripheral thrombosis, and the like. Thromboembolic events affect many people every year and may result in morbidity in patients throughout the word. Examples of morbidity include ischemia, loss of limb, angina pectoris, myocardial infarction, stroke, and pulmonary embolism. In some cases, death may result as a result of a thromboembolic event.

Common existing techniques for treating thromboembolic events include embolectomy, surgery, the use of therapeutic agents such as streptokinase or urokinase or other thrombolytic agents, or thrombectomy devices.

Other diseases or conditions may also result in occluded vessels. For example, blood vessels can spasm resulting in an occlusion which may restrict blood flow in the vessel. In the case of an artery in the brain, an irritant such as trauma, blood, a drug, or a seizure can result in spasm. The spasm can lead to functional narrowing of the artery and the resulting decrease in blood flow can cause a stroke.

Present treatments for occlusions caused by clots or spasm, or other caused for other reasons attempt to restore blood flow and can have challenges depending on the circumstances.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 shows an example of a stent retriever system that may be used for evaluating and treating thrombus.

FIG. 2 illustrates an example of an electro-pharmaceutical unit that may be included in the stent retriever system of FIG. 1.

FIG. 3A-3B show an example of an elongate shaft that may be included in the stent retriever system of FIG. 1.

FIG. 4 illustrates an example of a distal portion of a stent retriever that may be included in the stent retriever system of FIG. 1.

FIGS. 5A-5K illustrate an example of a method of evaluating and treating a thrombus.

FIGS. 6A-6D illustrate an example of a method of evaluating and treating vasospasm.

### DETAILED DESCRIPTION

Occlusion of a blood vessel is often caused by a clot, and this may be referred to as a thromboembolic event, and can result in disorders such as stroke, pulmonary embolism, peripheral thrombosis, and the like. Thromboembolic events affect many people every year and may result in morbidity in patients throughout the word. Examples of morbidity include ischemia, loss of limb, angina pectoris, myocardial infarction, stroke, pulmonary embolism. In some cases, death may result as a result of a thromboembolic event.

Common existing techniques for treating thromboembolic events include embolectomy, surgery, the use of therapeutic agents such as streptokinase or urokinase or other thrombolytic agents, or thrombectomy devices. These devices may provide varying clinical results depending on the blockage being treated. In certain instances, it may be beneficial to more accurately evaluate the type of blockage in order to provide a better, more customized treatment for the patient, and in some situations it may be beneficial to provide a treatment device that can provide different treatments or combination of treatments to the patient based on the type of blockage, thereby increasing safety of the procedure, reducing costs associated with having to use additional equipment, reducing procedure time, lowering inventory, etc. In some situations, the diagnostic or evaluation features may be combined with the therapeutic features in a single device.

In the situation where the blockage is a thrombus (also referred to herein as a clot), the thrombus may be a red thrombus or a white thrombus. Red thrombus is a clot rich in red blood cells which contains higher concentrations of hemoglobin, which is iron rich, while white thrombus is a clot rich in white blood cells, protein, and fibrous tissue. The treatment and removal of thrombus from a blood vessel may be more effective if the type of clot is known so that the treatment may be tailored to the specific type of clot as will be discussed in greater detail below.

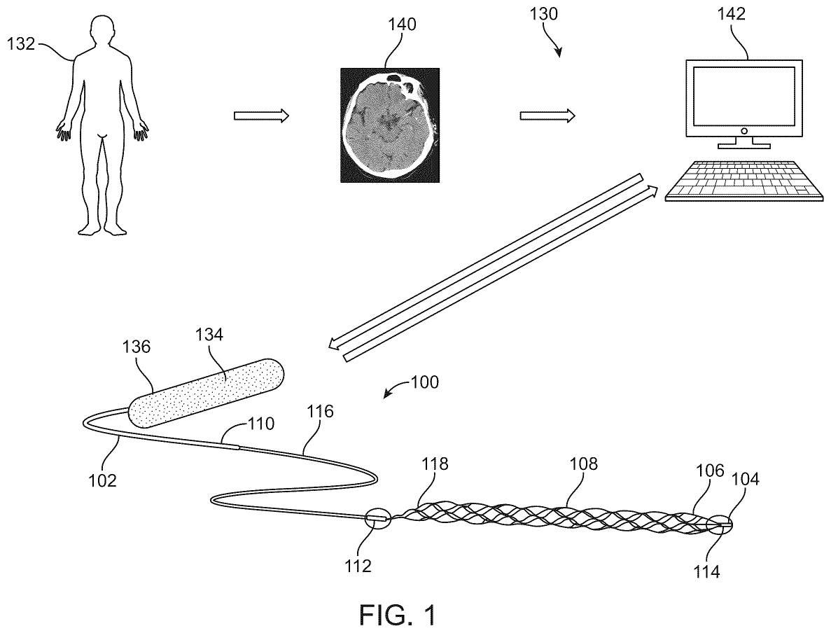

FIG. 1 shows a schematic overview of a device 100 and system 130 that may be used to evaluate and provide customized treatment for removal of clot from a patient 132. This device 100 may be referred to as a stent retriever. The devices, systems and methods disclosed herein are primarily directed at evaluation and treatment of thrombus in the arteries of the brain, but this is not intended to be limiting and the clots may be in any vessel in the body and the methods, systems, and apparatus disclosed herein may be used to treat a clot anywhere.

The system 130 for evaluating and treating a thrombus in a vessel may include an electropharmaceutical catheter 102 which has an elongate shaft 116 having a proximal end and a distal end. The distal end may be disposed in the patient and includes an expandable member 108 for removing clots while the proximal end may remain outside the patient and include a handle 134 for controlling or manipulating the device. A lumen may extend between the proximal and distal ends of the elongate shaft 116. An electro-pharmaceutical unit 136 may be coupled to the proximal end of the elongate shaft, and may be integral with the handle 134 or coupled therewith or disposed therein. An expandable member such as an expandable cage 108 may be coupled to distal end of the elongate shaft 116 with a coupling element 112 such as those disclosed in U.S. patent application Ser. No. 17/336,791 now U.S. Pat. No. 11,291,463; previously incorporated herein by reference. An example of the coupling element 112 maybe a collar that is crimped, soldered, bonded or otherwise coupled to the expandable cage 108 and the elongate shaft 116.

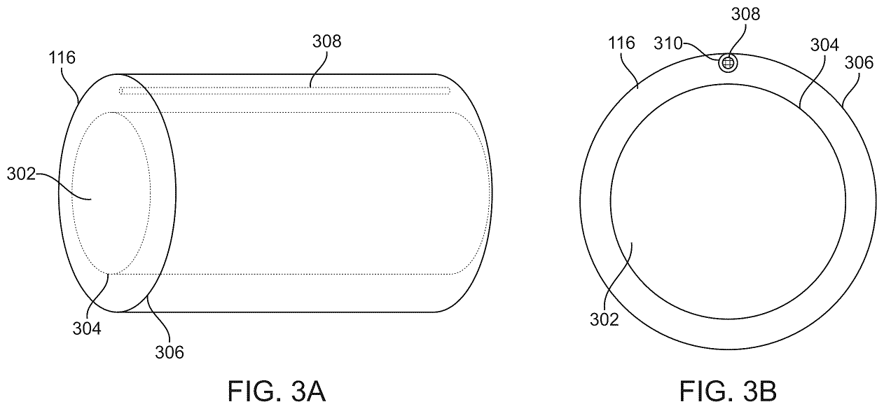

The elongate shaft 116 may be formed from a polymer or a metal tube and have one or more lumens. Additional details about elongate shaft 116 are disclosed below in FIG. 3.

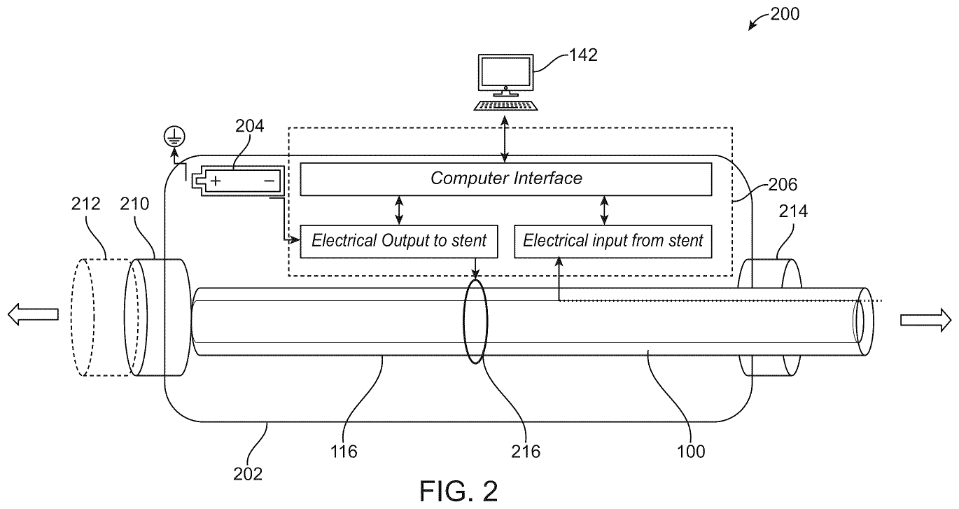

The electro-pharmaceutical unit 136 may be fixedly or releasably coupled to the proximal end of the elongate shaft. Additional details about the electro-pharmaceutical unit are discussed below in FIG. 2.

The expandable cage 108 has a collapsed configuration suitable for delivery through a vessel to the thrombus, and also has an expanded configuration where the cage will radially expand into and enmesh the thrombus so that the thrombus may be captured in the cage such that when the expandable cage is retracted proximally, the thrombus may be removed from the vessel to restore or improve patency of the vessel. The expandable cage may be balloon expandable or self-expanding or may be expandable based on temporary alteration of its electrical properties. In the case where the cage is self-expanding, an outer sheath 102 or outer catheter may have a distal portion 110 that can be slidably disposed over the shaft 116 and cage 108 to provide a constraint that prevents self-expansion. When the cage is advanced distally of the constraint (or when the constrain is retracted proximally relative to the cage), the cage becomes unconstrained and can self-expand into engagement with the thrombus. In the example where the cage is balloon expandable, the cage may be disposed over an inflatable balloon (not shown) which radially expands the cage when inflated. The outer sheath 102 is optional in the case of a balloon expandable cage and if used, provides protection to the expandable cage. Proximal or distal movement of the expandable sheath 102 in any example may be controlled by actuators such as levers, sliders, rotating wheels, etc. on handle 134 (not shown) and that are known in the art. The proximal portion of the expandable cage 108 may include an open proximal end 118 that is beveled, and the distal end of the cage may include a closed tapered conical distal end 106 that converges to a point 114 to help contain and trap any thrombus retrieved. Also, the distal end of the cage may include a lumen 104 that allows a guidewire to be slidably disposed through the cage and shaft 116 to facilitate over the wire delivery to a target treatment area. The expandable cage may have any number of geometries but in this example, the cage is formed from a series of interconnected closed cells. Examples of cell geometries which may be used are disclosed in U.S. patent application Ser. No. 17/336,791 now U.S. Pat. No. 11,291,463; previously incorporated herein by reference.

The system 130 for evaluating and treating thrombus may also include an imaging system 140 such as a computerized tomography system (CT), magnetic resonance imaging system (MRI), or any other imaging system known in the art for imaging thrombus in vessels of a patient such as in the brain. The imaging system 140 provides an image of the thrombus to a processor 142 so that the processor can characterize the thrombus and help evaluate the thrombus such as the size of the thrombus, location, type of thrombus, morphology, etc. The processor can then provide this information to the physician to help the physician determine the appropriate treatment plan. In some circumstances, the processor can provide suggested treatment plans to the physician based on evaluation of the imaging data. Once the processor or the physician, or both have reviewed the imaging data and characterization of the thrombus, the physician may begin treatment of the thrombus, or the processor may issue instructions to the electro-pharmaceutical unit 136 and help control the treatment. As the catheter 100 is used to treat the patient, sensors on the catheter may provide feedback to the processor 142 and the processor may make adjustments to the treatment in real time as the treatment is given to the patient, as will be discussed further below. Various treatment options are discussed in more detail below.

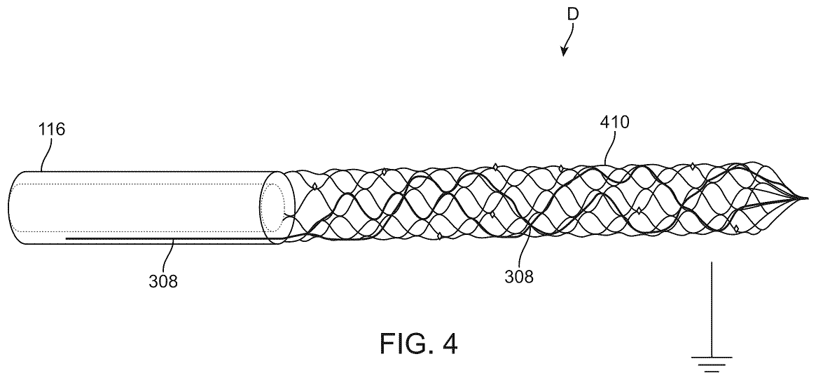

FIG. 2 illustrates more detail of an example of an electro-pharmaceutical unit 200 that may be used on the proximal end of the catheter, such as the catheter shown in FIG. 1. Here, a housing or handle 202 is sized and shaped to be easily held and manipulated by an operator's hand. The handle may house all of the electrical circuitry or other components, or some may be housed in the housing while other circuitry or components may be external to the housing such as coupled to the housing, or separate and discrete from the housing but operably coupled with the circuitry and components in or on the housing, e.g. by wireless connection such as near field communication or Blue Tooth connection, operably coupled with a cable, or other techniques known in the art. For example, a battery 204 (rechargeable or single use) may be disposed in the handle, and that can be used to power the device or provide electrical current as part of the evaluation and treatment as will be explained below. In other examples, the power for the catheter may be supplied by an external power source such as an external battery or the catheter may be plugged into a power supply such as the house mains (e.g. 120 or 240 volt wall outlet) which is not shown in FIG. 2. An interface circuit 206 sends and receives information to/from the processor 142 to help evaluate and treat the thrombus. The interface circuit 206 receives electrical information from the sensor on the expandable cage (best seen in FIG. 4) and transmits it to the computer 142, and instructions from the computer are transmitted to the catheter 100 to control electrical output from the catheter to the thrombus. In some examples, the elongate shaft 116 is conductive and electrically coupled with a conductor element 216 that is also electrically coupled with the interface circuit 206, therefore the computer interface 206 allows the elongate shaft 116 to deliver an electrical current from the battery 204 or power source to the thrombus, and/or receive electrical information about the clot sensed by the expandable cage on the catheter distal end. The elongate shaft 116 may be formed from a conductive metal such as nitinol or stainless steel, or the elongate shaft may be formed from a conductive polymer, or combinations of metals and polymers. Therefore the battery or power source 204 is electrically coupled to the elongate shaft 116 via the computer interface.

The distal end of the catheter shaft 116 may include a collar 214 or other coupling element for joining the expandable cage (best seen in FIG. 1) with the elongate shaft 116. The proximal end of the elongate shaft may extend out of the proximal end of the handle and may include a connector 210 such as a Luer fitting (not shown) or another connector to allow the catheter to be releasably coupled to other tubing 212, another device, or fluidly coupled to a source or reservoir of a therapeutic agent or other fluid (e.g. saline, heparin, thrombus lysing or softening agents, etc.), thus the therapeutic agent may be delivered from the source through the lumen and the drug is delivered at the distal end of the elongate shaft into the area of the thrombus. Examples of drugs may be thrombolytics (e.g. streptokinase or urokinase) that break up clots, or other therapeutic agents that soften the clot, or otherwise have a desired therapeutic effect on the clot. Additional examples include thrombolytics such as alteplase and tenecteplase for dissolving thrombus; calcium channel blockers such as nimodipine for relieving vasospasm; anti-platelet drugs such as aspirin, clopidogrel for preventing platelet clot formation after treatment; enzymes such as elastase to soften white clot; and neuroprotection drugs such as magnesium and nerinitide. This list of therapeutic agents is not intended to be limiting, and a physician may use any therapeutic agent needed for a positive clinical outcome.

FIGS. 3A-3B show an example of the elongate shaft 116 of a catheter such as the one illustrated in FIG. 1 that may be used to evaluate and treat thrombus. FIG. 3A shows a side view of the elongate shaft and FIG. 3B shows a cross-section of the elongate shaft taken orthogonally to the longitudinal axis of the elongate shaft. Here, the elongate shaft 116 has a single lumen 302 which extends from the proximal end of the elongate shaft to the distal end of the elongate shaft. As previously discussed above, the elongate shaft 116 is coupled to the electro-pharmaceutical unit and a connector such as a Luer hub may be coupled to the lumen so that therapeutic agents may be delivered through the lumen to the thrombus. The electro-pharmaceutical unit and connector are best seen in FIGS. 1-2.

The elongate shaft 116 may include an inner shaft 304 and an outer shaft or coating 306 disposed over the inner shaft 304. The inner shaft 304 may be electrically conductive as previously discussed so that an electrical current may be delivered from the electro-pharmaceutical unit, along the elongate shaft to the thrombus via the expandable cage. Examples of conductive elongate shafts include shafts formed from metals such as nitinol or stainless steel, or conductive polymers, and combinations of metals and polymers. The outer coating or outer shaft 306 may be a layer of insulation over the inner shaft 304. Additionally, in some examples a separate wire (not shown) may be used to deliver the electrical current from the electro-pharmaceutical unit to the thrombus via the expandable cage in addition to, or instead of using an inner conductive shaft.

A sensor wire 308 may be disposed over the inner shaft or in a wall of the elongate shaft and run from the electro-pharmaceutical unit to the expandable cage on the catheter. The sensor wire 308 may be disposed in a second lumen 310 in the wall of the elongate shaft. The sensor wire 308 may include insulation to prevent interference between the sensor signal carried on the sensor wire and any electrical current being delivered along the elongate shaft to the thrombus. The insulation may be provided by any material, technique or any other ways known in the art. An example of an insulation material that may be used to insulate any of the wires in any of the examples of devices disclosed herein includes methacryloyloxyethyl phosphorylcholine (MPC). The sensor wire may be a high electrical conductance wire (e.g. low impedance wire) so that any electrical sensor signal is transmitted without distortion or significant attenuation. This may be referred to as performing an in-vivo immediate electrical biopsy of the clot.

Additional details about delivery of an electrical current from the catheter to the thrombus, and sensing characteristics of the thrombus with the sensing wire are discussed in more detail below.

FIG. 4 illustrates an example of a distal end D of a catheter that may be used to evaluate and treat of a thrombus, such as the one shown previously in FIG. 1.

The distal end of the elongate shaft 116 is coupled to the expandable cage 410 which was previously described above in FIG. 1. Thus, the structure and function of the expandable cage is the same as in FIG. 1. The sensor wire 308 runs along the elongate shaft 116 and exits the distal end of the elongate shaft and then may be coupled to the expandable cage 410. The sensor wire may extend along or around the expandable cage in any desired pattern. Here, the sensor wire extends helically around the expandable cage like a corkscrew along the entire length or partial length of the expandable cage and the distal end of the sensor wire is coupled to the distal end (or anywhere desired) of the expandable cage. The sensor wire may be insulated along its length to prevent interference with other electrical elements on the catheter, and sections along the sensor wire may have the insulation removed to allow electrical contact with the thrombus. The sensor wire may be a single wire which can measure the impedance of the thrombus (with the body serving as ground), or the sensor wire may be two wires side by side for measuring impedance of the thrombus along the length of the expandable cage. The sensor wire may be a single wire that is split into two or more wires that are disposed over the expandable cage to provide additional sensing positions. In the example where a single wire is used to measure impedance, the body may be used as the ground or return path. Here a grounding plate such as a Bove plate may be placed under the patient's body in electrical contact with the body to serve as the return path. In the example where two or more wires are used to measure impedance, one of the wires may be used to deliver current to the patient for impedance measurement and one wire may be used as the return path.

Additionally, the elongate shaft may be electrically conductive as previously described and therefore, an electrical current may be delivered from the electro-pharmaceutical unit, along the elongate shaft to the expandable cage which is electrically coupled with the elongate shaft. Thus, the expandable cage may be used to deliver an electrical current to the thrombus. Delivery of a current to the thrombus with the expandable cage is isolated from the electrical sensing wire which may measure impedance of the thrombus, so the two functions do not interfere with one another during operation. Various positions along the expandable cage may be insulated so that the electrical charge is only delivered from selected positions along the expandable cage (e.g. nodes). The body may serve as the ground or return path for the electrical circuit and a Bove plate may be placed under the patient's body to create the return path as indicated by the ground symbol in FIG. 4. Or, in other examples, two wires may be provided with one delivering the electrical charge and one wire serving as the return path.

Adequate slack in the wire or wires and adequate attachment of the wire or wires to the expandable cage is accomplished using techniques known in the art in order to accommodate the radial expansion and collapse of the expandable cage.

In use, the evaluation and treatment catheter may be used to treat a stroke patient with a thrombus blocking a vessel in the brain. An image of the thrombus is obtaining using imaging techniques known in the art and the image is analyzed to characterize the thrombus such as size, shape, length, morphology, type of clot, etc. This information may be determined by a physician or using a computer processor and is used to help determine treatment of the thrombus.

Additionally, a catheter such as the one disclosed herein may be delivered to the thrombus to further evaluate and treat the thrombus. For example, once the catheter is delivered to the thrombus, the expandable cage may be radially expanded into engagement with the thrombus to enmesh the thrombus. The sensor wire may be used to measure impedance of the thrombus. Thrombus may be a red clot or a white clot. Red clots are rich in red blood cells which have hemoglobin and therefore have high iron concentrations which results in a lower impedance than a white clot. White clots are rich in white blood cells, fiber and therefore are not as conductive as red clots and so they have higher impedance. Measuring impedance allows the physician to help determine the type of clot present in the patient. This may be referred to as performing an in-vivo immediate electrical biopsy of the clot.

If the clot is a white clot, a therapeutic agent may be delivered using the evaluation and treatment catheter to help soften or lyse the white clot which is tough, hard, and fibrous. Examples of softening drugs that may be used include thrombolytics such as alteplase and tenecteplase for dissolving thrombus; anti platelet drugs such as aspirin, clopidogrel for preventing platelet clot formation after treatment; and enzymes such as elastase to soften white clots. Again, this list of softening drugs is not intended to be limiting and other drugs may also be used.

Once softened, the expandable cage may be used to help remove the clot by proximally retracting the expanded cage which is enmeshed with the clot.

In the case of red clots, they are softer than the white clots and therefore do not necessarily require a therapeutic agent to soften them but optionally may use a softener or lysis agent. However, the evaluation and treatment catheter may also be used to deliver a negative charge to the expandable cage and the negative charge may help the iron rich red clot to stick to the expandable cage which is enmeshed with the red clot, and thus when the expandable cage is proximally retracted, the clot is more likely to stick to the expandable cage and be removed.

For either red or white clots, the catheter may also be used to deliver other therapeutic agents that will help with restoring patency to the vessel, or optionally deliver a charge to the clot, or sense conductivity of the clot to help diagnose clot type.

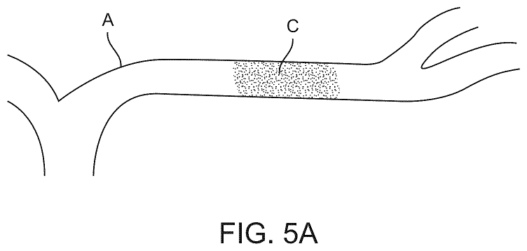

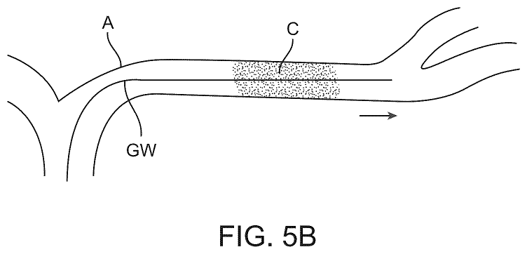

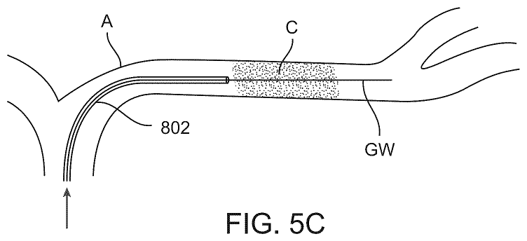

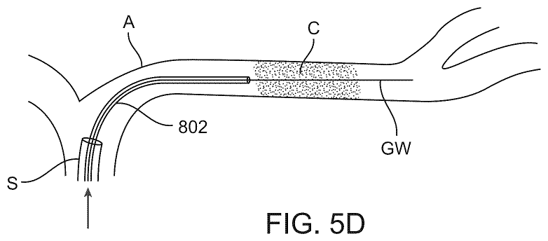

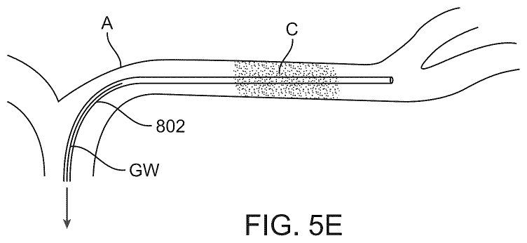

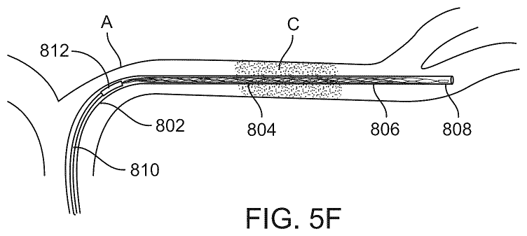

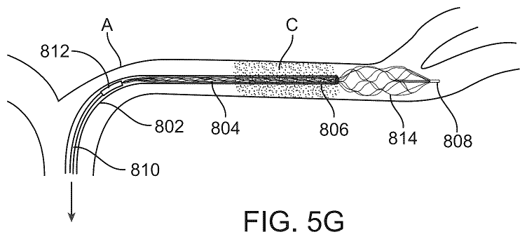

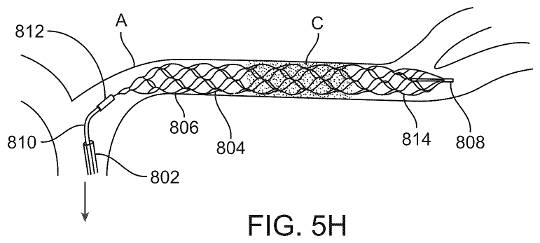

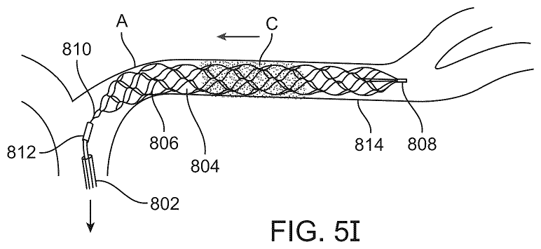

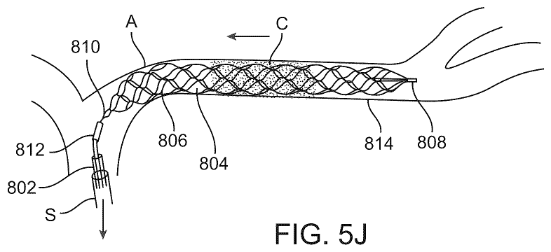

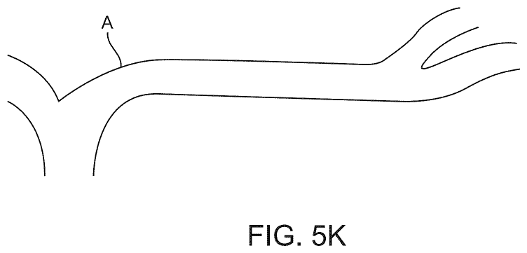

FIGS. 5A-5K illustrate an example of a method for treating a patient suffering a stroke due to a blood clot in an artery of the brain. This is not intended to be limiting and one of skill in the art will appreciate that the devices and methods disclosed herein may be used to treat clots or obstructions in other vessels of the body or other anatomical locations. Any of the examples of evaluation and treatment catheters disclosed herein may be used in this example of a method of treatment.

In FIG. 5A, an artery A of the brain is shown with a blood clot C occluding the vessel preventing or limiting oxygenated blood from flowing past the blockage thereby causing a stroke due to ischemia. The clot may fully or partially occlude blood flow through the vessel.

In FIG. 5B, a guidewire GW is introduced percutaneously into a blood vessel typically an artery, and advanced through the vessel, through the clot and distal of the clot. The guidewire may be introduced percutaneously into a vessel using standard procedures such as the Seldinger procedure or using a surgical cut down. Examples of access points may include the groin, the wrist, the neck, etc. and the guidewire may be advanced in any artery (e.g. femoral artery, carotid artery, radial artery, etc.) or any vessel such as a vein using known access techniques.

Once the guidewire is in place, it acts as a rail over which a microcatheter may be advanced to the treatment site. In FIG. 5C, a microcatheter 802 with a lumen is advanced distally in the affected artery A over the guidewire GW to the clot C. The microcatheter may be a single lumen catheter that provides a tunnel through which a stent retriever catheter (sometimes also referred to herein as a clot retriever or device for removing obstructions) may be advanced through the vasculature to the treatment site. The size of the microcatheter may be selected based on the vessel being treated. The stent retriever may be any of those disclosed herein.

FIG. 5D shows optional use of a sheath S. Here, after the guidewire is inserted, a sheath (e.g. a guide sheath) may be advanced over the guidewire GW. The microcatheter is then advanced over the guidewire GW and through a lumen of the sheath S until the microcatheter is adjacent or abuts the clot C. Thus, if a sheath is used, FIG. 5D replaces FIG. 5C. Once the microcatheter has been properly advanced and positioned, the sheath may either remain in place, be retracted proximally to move it out of the way but remain in the vessel, or the sheath may be entirely removed from the vessel. The microcatheter remains disposed adjacent the clot.

In FIG. 5E, the microcatheter 802 is advanced through the artery A so that its distal tip passes over the guidewire GW, through the clot C and is distal of the clot C. Once the microcatheter is delivered to a desired position, the guidewire GW is retracted proximally and may be removed from the patient.

Once the microcatheter has been positioned, a stent retriever catheter such as any of those disclosed herein may introduced through a port of an introducer (not shown) at the vascular access point and advanced through the microcatheter toward the treatment region. An outer sheath (not shown here but best seen in FIG. 1) may be disposed over the stent-retriever catheter for packaging and shipping purposes as well as to constrain the expandable cage. Thus, once the stent retriever has been inserted into the microcatheter, the outer sheath on the stent retriever may be proximally retracted and removed from the expandable cage. The microcatheter then constrains and prevents the expandable cage from self-expanding.

FIG. 5F shows the clot retriever catheter 806 disposed in the microcatheter 802 and traversing the clot C. The clot retrieval device 806 is shown including the distal portion of the expandable cage traversing the clot C, the expandable capture cage 804, elongate shaft 810, optional proximal radiopaque marker or collar 812 and optional distal radiopaque marker or collar 808. The clot retriever catheter 806 may be any of the examples disclosed herein.

The micro catheter 802 not only provides a channel for delivering the clot retrieval catheter 806 but also provides a constraint that holds the expandable capture cage 804 in a collapsed configuration during delivery. Optional proximal and distal radiopaque markers 808, 812 allow the operator to visualize the position of the device and ensure that the expandable capture cage is disposed along the entire length of the clot C. Elongate shaft 810 may be a guidewire coupled to the expandable capture cage and allows the operator to push or pull the device along the artery A and through the microcatheter relative to the clot C. The elongate shaft may also be any of the examples of elongate shaft disclosed herein, and they may be braided or otherwise reinforced or otherwise manufactured to provide the desired pushablity, or the elongate shaft may be a combination of a guidewire and any of the other elongate shafts disclosed herein in order to provide a pushable shaft that can conduct current or other electrical signals to/from the expandable cage and the electro-pharmaceutical unit. The expandable capture cage may include any or all permutations or combinations of capture cage features described in any example in this specification or otherwise incorporated by reference.

Once the capture cage 804 has been properly positioned relative to the clot C, the microcatheter 802 is retracted proximally as shown in FIG. 5G while the clot retrieval catheter remains stationary or is advanced slightly out of the microcatheter. As the microcatheter 802 is removed from the expandable capture cage 804, it self-expands 814 into engagement with the clot C and the vessel wall. Because the walls of the capture cage are formed from closed cells, the walls of the cage are porous with apertures extending through the wall creating a mesh-like cage wall which can expand into the clot C and enmesh the clot so that the clot is disposed inside the cage and the struts of the cage are outside or mostly outside of the clot. FIG. 5G shows partial expansion of the distal portion of the cage that extends beyond the clot.

In FIG. 5H, further proximal retraction of the microcatheter 802 unsheathes the entire capture cage so it can self-expand and enmesh the clot C thereby integrating the clot with the expandable cage. The capture cage may self-expand into apposition with the walls of the vessel. The entire clot C is now substantially in the expanded capture cage 814.

Any of the sensing or therapeutic techniques described above (e.g. measuring impedance, applying a negative charge to the expandable cage, delivering a therapeutic agent, etc.) may be performed at this point, or at any other desired point of this example of a method.

FIG. 5I shows that once the cage 814 has self-expanded and enmeshes the clot C, and after any of the sensing and/or therapeutic techniques described above (e.g. measuring impedance, applying a negative charge to the expandable cage, delivering a therapeutic agent, etc.) have been performed, the entire clot retrieval device 806 is proximally retracted by pulling on the elongate shaft 810 which extends proximally outside of the patient's body so that an operator may grasp and manipulate the proximal end. Proximal retraction of the catheter also carries the enmeshed clot C with the cage 814 as it is retracted proximally. The catheter 806 and clot along with the microcatheter 802 are then retracted proximally until they are removed from the patient. The expandable cage 814 may be retracted proximally so that the proximal end of the expandable cage approximates the distal end of the microcatheter, or the two ends may remain separated from one another. Both the microcatheter and the elongate shaft 810 of the clot retrieval catheter may be retracted proximally together or individually.

FIG. 5J shows the optional sheath S that may have been used to help deliver the clot retrieval catheter (see FIG. 5D). After the clot has been enmeshed in the cage 814, the microcatheter and the cage 814 may optionally be retracted proximally into the sheath S and everything removed from the patient. Or optionally, the microcatheter and cage 814 may be retracted proximally partially and adjacent the distal end of the sheath S, but not necessarily all the way into the sheath S and then everything may be removed from the patient.

FIG. 5K shows the artery after the clot, microcatheter, optional sheath, and clot removal device have been removed from the artery A. The vessel is now patent again once the obstruction has been removed and tissue downstream of the clot will now receive oxygenated blood.

### Other Applications—Vasospasm

As previously mentioned above, blood vessels may go into spasm, and this can restrict blood flow which can result in ischemia including a stroke in the brain. Any of the examples of diagnostic and therapeutic devices disclosed herein may be used to treat blockages such as those caused by a thrombus, but also may be used to treat vasospasm. Other uses for the devices and methods disclosed herein are also contemplated and are not limited to clot treatment or vasospasm.

As an example, sometimes an artery in the brain goes into a spasm due to an irritant including trauma, blood, drug, seizure, etc. This spasm leads to functional narrowing of the artery and can decrease blood flow and cause a stroke. The treatment for such cases is to relieve the spasm. There are several ways to accomplish this including—mechanically inflating an expandable member such as a balloon or radially expanding an expandable stent-like structure inside the narrowed segment to restore vessel patency, or delivery of a drug to relieve the spasm (for example, a calcium channel blocker). Examples of the present device disclosed herein may be used to mechanically support the spasm or deliver a drug with the catheter to the treatment area. Radial expansion of the expandable cage may provide adequate mechanical support to the vessel to alleviate the vasospasm. Additionally, examples of the devices disclosed herein may also be used to deliver electrical stimulation to the spasm in order to relax the muscles in the vessel such as an artery causing the spasm. Electrical stimulation may be combined with mechanical support provided by the expandable cage and/or with use of the devices disclosed herein to delivery a therapeutic agent to the vasospasm.

Therefore, any of the examples of the devices disclosed herein may be used to deliver one or more of the following therapies to the spasm to help restore blood flow.

The expandable cage may serve as a mechanical support to help force open the spasm and hold it open until the spasm is alleviated. Thus, any of the devices described herein are delivered to the region of spasm and then the expandable cage is expanded to engage and open the restricted area to help restore flow.

The device may be used to deliver a therapeutic agent either through a lumen in the device, or the therapeutic agent may be carried on the expandable cage and eluted therefrom. This allows site specific delivery of the drug to the spasm. Examples of drugs have previously been discussed above.

Any of the examples of devices may also be used to measure the electrical potential of the artery or other vessel in a normal, unaffected region as well as the narrowed region caused by spasm and consequently the device may be used to apply an opposite measured charge to relieve the spasm. Therefore, in the examples above, instead of delivering a charge to a thrombus, the charge is delivered to the vessel experiencing spasm to alleviate the narrowing.

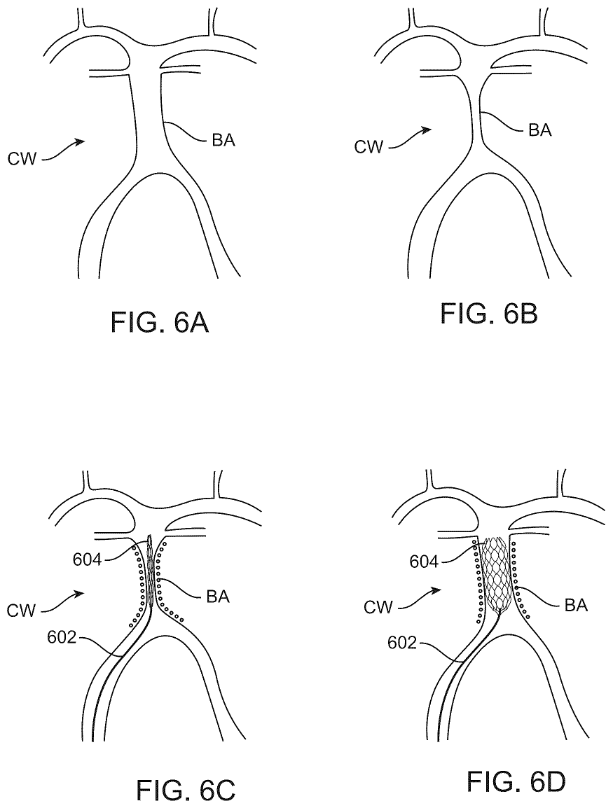

FIGS. 6A-6D illustrate the use of any of the examples of devices disclosed herein to treat a spasm in an intracranial artery or vein. This is not intended to be limiting and this example of a treatment may be used in any vessel or any other portion of the body.

FIG. 6A shows a portion of the normal anatomy of the Circle of Willis, CW which is a circulatory anastomosis of the major arteries supplying blood to the brain. Here, a part of the Circle of Willis including the basilar artery BA is shown. The basilar artery is one of the major arteries supplying blood to the posterior brain and brainstem. While there is some tapering of the vessel in the antegrade blood flow direction, there are no narrow or significantly reduced diameters and side branches or bifurcations remain patent.

FIG. 6B shows a small segment of the basilar artery BA undergoing vasospasm with significant narrowing of the vessel, thereby reducing blood flow which can result in stroke in the patient due to inadequate oxygen being delivered to the brain. This may occur in a variety of disease states and is often accompanied with severe neurological symptoms suggestive of an ischemic stroke.

FIG. 6C shows an example where any of the devices 602 disclosed herein is delivered through the vessel into the region of spasm. Once in position which may be verified using fluroscopy or any other imaging technique, a treatment regime may be delivered. The treatment may include mechanical support from the expandable cage 604 once expanded, delivery of a drug from the catheter device 602 to alleviate the spasm (e.g. a calcium channel blocker or sodium channel blocker) which can alleviate spasm caused by hypertonic smooth muscles in the wall of the artery or vein, or delivery of an electrical current from the catheter device 602 to alleviate the spasm. Electric potential at the inner wall of the artery or the vein may be measured by the expandable cage. Such electric potential measurements may inform the physician about the electrical state of the smooth muscles in the arterial or venous walls. Once a discrepancy in electrical potential is identified (when compared to a normal segment before or after the vasospastic segment), diagnosis and subsequent management of the vasospasm can be more effective and more personalized.

FIG. 6D shows the basilar artery returning to its normal diameter after the spasm has been treated by any one or combination of the treatment regimes disclosed above. The expandable cage if expanded may be collapsed and the device may be removed from the patient once blood flow has been restored, leaving a patent blood vessel.

Because the examples of devices disclosed herein measure and provide information to a physician about the characteristics of the vessel being treated, e.g. nature of the thrombus or vasospasm, real time feedback may be provided to the physician thereby allowing the physician to modify treatment as the vessel characteristics change. For example, as a thrombus changes during treatment or as vasospasm changes, the treatment may be adjusted such as by altering drugs delivered, modifying support provided by the expandable cage, or by modifying the electrical stimulation provided by the device. Therefore, optimized, real-time feedback based treatment (e.g. electrical stimulation) may be provided to the patient to relieve thrombus or vasospasm. In some examples, the treatment is manually modified by the physician or technical, while in other examples the treatment is automatically modified by the device or system itself.

### NOTES AND EXAMPLES

The following, non-limiting examples, detail certain aspects of the present subject matter to solve the challenges and provide the benefits discussed herein, among others.

Example 1 is a device for treating a thrombus or a spasm in a blood vessel of a patient, the device comprising: an elongate shaft having a proximal end, a distal end, and a lumen extending therebetween; an electro-pharmaceutical unit coupled to the proximal end of the elongate shaft, wherein the electro-pharmaceutical unit is configured to facilitate evaluation and treatment of the thrombus or the spasm; an expandable cage coupled to the distal end of the elongate shaft, the expandable cage having a collapsed configuration for delivery through a vessel to the thrombus or the spasm, and an expanded configuration for enmeshing the thrombus or supporting the spasm; and an electrical element coupled to the expandable cage, the electrical element configured to sense a characteristic of the thrombus or the spasm or to provide a treatment to the thrombus or the spasm.

Example 2 is the device of Example 1, wherein the electro-pharmaceutical unit is releasably coupled to the proximal end of the elongate shaft.

Example 3 is the device of any of Examples 1-2, wherein the electro-pharmaceutical unit comprises electronic circuitry operatively coupled to a processor, the electronic circuitry configured to facilitate evaluation and treatment of the thrombus or the spasm.

Example 4 is the device of any of Examples 1-3, wherein the electro-pharmaceutical unit further comprises a housing configured to house the electronic circuitry.

Example 5 is the device of any of Examples 1-4, wherein the electrical element comprises a conductive wire coupled to the expandable cage, the conductive wire configured to measure impedance of the thrombus or measure electrical potential in the spasm or adjacent thereto.

Example 6 is the device of any of Examples 1-5, wherein the elongate shaft is formed from a conductive material, the elongate shaft forming at least a portion of the electrical element and configured to deliver an electrical charge to the expandable cage.

Example 7 is the device of any of Examples 1-6, wherein the device is configured to provide optimized real-time feedback based electrical stimulation to relieve the vasospasm.

Example 8 is a system for treating a thrombus or a spasm in a patient, the system comprising: the device of any of Examples 1-7; an imaging system configured to provide an image of the thrombus or the spasm; and a processor configured to evaluate the image of the thrombus or the spasm and provide a characterization of the thrombus or the spasm, and wherein the processor controls the electro-pharmaceutical unit to deliver a customized treatment to the thrombus or the spasm.

Example 9 is the system of Example 8, further comprising a reservoir of a therapeutic agent fluidly coupled with the lumen, the therapeutic agent configured to lyse, soften, or otherwise provide a desired effect on the thrombus, or wherein the therapeutic agent is configured to alleviate the spasm.

Example 10 is a method for evaluating or treating a thrombus or a spasm in a vessel of a patient, the method comprising: advancing a catheter with an elongate shaft and an expandable cage through a vessel toward the thrombus or the spasm; sensing a characteristic of the thrombus or the spasm with the catheter; and based on the sensed characteristic, evaluating the thrombus or the spasm and determining a treatment for removing or reducing the thrombus, or for alleviating the spasm, and restoring patency of the vessel.

Example 11 is the method of Example 10, wherein the sensing comprises measuring an impedance of the thrombus with the catheter to characterize the thrombus or measuring a potential in the spasm or adjacent thereto.

Example 12 is the method of any of Examples 9-11, wherein the treatment comprises radially expanding the expandable cage to enmesh the thrombus, the method further comprising proximally retracting the expandable cage to remove the thrombus from the vessel, or wherein the treatment comprises radially expanding the expandable cage to support the spasm.

Example 13 is the method of any of Examples 9-12, wherein the treatment comprises electrically charging the expandable cage to facilitate capture of the thrombus with the expandable cage, or to electrically stimulate the spasm.

Example 14 is the method of any of Examples 9-13, wherein the treatment comprises delivering a therapeutic agent from the catheter to the thrombus or the spasm, the therapeutic agent configured to lyse, soften, alleviate spasm, or otherwise provide a desired effect on the thrombus or spasm.

Example 15 is the method of any of Examples 9-14, further comprising imaging the thrombus or the spasm and evaluating an image of the thrombus or the spasm to determine a treatment for removing the thrombus or alleviating the spasm.

Example 16 is the method of any of Examples 9-15, further comprising providing optimized real-time feedback based electrical stimulation to relieve the vasospasm.

In Example 17, the apparatuses, systems or methods of any one or any combination of Examples 1-16 can optionally be configured such that all elements or options recited are available to use or select from.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

1. A device for treating a thrombus or a spasm in a blood vessel of a patient, the device comprising:
an elongate shaft having a proximal end, a distal end, and a lumen extending therebetween;
an electro-pharmaceutical unit coupled to the proximal end of the elongate shaft, wherein the electro-pharmaceutical unit is configured to facilitate evaluation and treatment of the thrombus or the spasm;
an expandable cage coupled to the distal end of the elongate shaft, the expandable cage having a collapsed configuration for delivery through a vessel to the thrombus or the spasm, and an expanded configuration for enmeshing the thrombus or supporting the spasm; and
an electrical element coupled to the expandable cage, the electrical element configured to sense a characteristic of the thrombus or the spasm or to provide a treatment to the thrombus or the spasm.

2. The device of claim 1, wherein the electro-pharmaceutical unit is releasably coupled to the proximal end of the elongate shaft.

3. The device of claim 1, wherein the electro-pharmaceutical unit comprises electronic circuitry operatively coupled to a processor, the electronic circuitry configured to facilitate evaluation and treatment of the thrombus or the spasm.

4. The device of claim 3, wherein the electro-pharmaceutical unit further comprises a housing configured to house the electronic circuitry.

5. The device of claim 1, wherein the electrical element comprises a conductive wire coupled to the expandable cage, the conductive wire configured to measure impedance of the thrombus or measure electrical potential in the spasm or adjacent thereto.

6. The device of claim 1, wherein the elongate shaft is formed from a conductive material, the elongate shaft forming at least a portion of the electrical element and configured to deliver an electrical charge to the expandable cage.

7. The device of claim 1, wherein the device is configured to provide optimized real-time feedback based electrical stimulation to relieve the vasospasm.

8. A system for treating a thrombus or a spasm in a patient, the system comprising:
the device of claim 1;
an imaging system configured to provide an image of the thrombus or the spasm; and
a processor configured to evaluate the image of the thrombus or the spasm and provide a characterization of the thrombus or the spasm, and wherein the processor controls the electro-pharmaceutical unit to deliver a customized treatment to the thrombus or the spasm.

9. The system of claim 8, further comprising a reservoir of a therapeutic agent fluidly coupled with the lumen, the therapeutic agent configured to lyse, soften, or otherwise provide a desired effect on the thrombus, or wherein the therapeutic agent is configured to alleviate the spasm.

10. A method for evaluating or treating a thrombus or a spasm in a vessel of a patient, the method comprising:
advancing a catheter with an elongate shaft and an expandable cage through a vessel toward the thrombus or the spasm;
sensing a characteristic of the thrombus or the spasm with the catheter; and
based on the sensed characteristic, evaluating the thrombus or the spasm and determining a treatment for removing or reducing the thrombus, or for alleviating the spasm, and restoring patency of the vessel.

11. The method of claim 10, wherein the sensing comprises measuring an impedance of the thrombus with the catheter to characterize the thrombus or measuring a potential in the spasm or adjacent thereto.

12. The method of claim 10, wherein the treatment comprises radially expanding the expandable cage to enmesh the thrombus, the method further comprising proximally retracting the expandable cage to remove the thrombus from the vessel, or wherein the treatment comprises radially expanding the expandable cage to support the spasm.

13. The method of claim 10, wherein the treatment comprises electrically charging the expandable cage to facilitate capture of the thrombus with the expandable cage, or to electrically stimulate the spasm.

14. The method of claim 10, wherein the treatment comprises delivering a therapeutic agent from the catheter to the thrombus or the spasm, the therapeutic agent configured to lyse, soften, alleviate spasm, or otherwise provide a desired effect on the thrombus or spasm.

15. The method of claim 10, further comprising imaging the thrombus or the spasm and evaluating an image of the thrombus or the spasm to determine a treatment for removing the thrombus or alleviating the spasm.

16. The method of claim 10, further comprising providing optimized real-time feedback based electrical stimulation to relieve the vasospasm.
